(19) United States
(12) Patent Application Publication
(10) Pub. No.: US 2025/0319151 A1
(43) Pub. Date: Oct. 16, 2025

(54) COSMETIC ACTIVE FOR HAIR AND SCALP TREATMENT TO STIMULATE HAIR GROWTH, ANTI-AGING OF HAIR AND PREVENTION OF PREMATURE HAIR LOSS

(71) Applicant: Henkel AG & Co. KGaA, Duesseldorf (DE)

(72) Inventors: Sabine Gruedl, Erkelenz (DE); Thomas Welss, Duesseldorf (DE); Gudrun Heinen, Duesseldorf (DE); Melanie Ludwig, Grevenbroich (DE); Camille Grosjacques, Stamford, CT (US); Volker Scheunemann, Luneburg (DE)

(21) Appl. No.: 19/169,645

(22) Filed: Apr. 3, 2025

Related U.S. Application Data
(60) Provisional application No. 63/633,301, filed on Apr. 12, 2024.

(51) Int. Cl. A61K 38/01; A61K 36/185; A61K 36/48; A61K 36/81; A61K 36/899; A61P 17/14

(52) U.S. Cl. CPC A61K 38/011; A61K 36/3482; A61K 36/48; A61K 36/81; A61K 36/899; A61P 17/14

(57) ABSTRACT

Disclosed is the use of hydrolyzed plant-based protein to promote hair growth and reduce hair loss. Hydrolyzed plant-based protein has been found to prolong the active growth phase in hair models, promote gene expression of members of the hair keratin gene family, and increase the release of growth factors.

### CROSS-REFERENCE TO RELATED APPLICATION

This application claims the benefit of U.S. Provisional Application No. 63/633,301, filed Apr. 12, 2024, the content of which is incorporated herein by reference in its entirety.

### BACKGROUND

The structure of hair includes the hair shaft, the portion located outside the skin surface, and the hair follicle, the portion located below the skin surface. The follicle is the structure from which hair grows. The follicle includes the hair bulb containing dermal papilla which actively produces hair. Growth of the hair follicle follows a cycle including an anagen (growth) phase, catagen (transition) phase, and telogen (resting) phase. A final phase (exogen) involves shedding old hair.

Hair ageing leads to hair strands becoming smaller, a decrease in hair production, and hair loss. Promotion of the growth phase of the hair follicle cycle would in turn promote hair growth and reduce hair loss.

Known chemical interventions for treating hair loss include oral and topical treatments using minoxidil or finasteride. Still other approaches for treating hair loss include herbal extracts. Extreme interventions include hair transplants.

There remains a need in the art for new convenient and facile approaches to promote hair growth and prevent premature hair loss.

### SUMMARY

Disclosed, in various embodiments, is a method of promoting hair growth and reducing hair loss comprising contacting hair and/or scalp of an individual with an effective amount of a hydrolyzed plant-based protein.

In another embodiment, a method of promoting gene expression of a member of the hair keratin gene family, increasing release of growth factors, or a combination thereof, comprises contacting hair and/or scalp of an individual with an effective amount of a hydrolyzed plant-based protein.

In another embodiment, a method of prolonging the active growth phase of hair growth, comprises contacting hair and/or scalp of an individual with an effective amount of a hydrolyzed plant-based protein.

These and other features and characteristics are more particularly described below.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It has been surprisingly found that treatment of scalp and hair follicles with hydrolyzed proteins of vegetable origin leads to a positive impact on hair health, including improved longitudinal growth and prolongation of the active growth phase. Treatment with these protein hydrolysates further provides for the stimulation of hair keratins and growth factors. It was surprisingly found that application of hydrolyzed plant-based protein provided stimulation of gene expression of hair keratins, in particular KRT33A and KRT34, in hair follicle models. Additionally, an increased release of growth factors was detected.

Disclosed is the use of a hydrolyzed plant-based protein for the stimulation of the structural components of the hair and for the protection against premature hair loss. The hydrolyzed plant-based protein not only provides positive effects on the hair fiber, but surprisingly provides positive effects on the hair follicle, hair growth, and prolongation of the active growth phase of hair.

In an embodiment, a method of stimulating the growth of hair and reducing hair loss by inducing the anagen phase comprises contacting the hair and scalp with hydrolyzed plant-based protein. Use of these proteins further provides prolongation of the anagen phase, the active growth phase. The method provides an improvement of the hair growth cycle, allowing the hair shaft to grow longer.

In a further embodiment, a method of promoting gene expression of a member of the hair keratin gene family, increasing release of growth factors, or a combination thereof comprises contacting the hair and scalp with hydrolyzed plant-based protein. Use of these proteins has been found to stimulate gene expression of hair keratins, e.g. KRT33A and KRT34, in hair follicle models. It was further discovered that use of the hydrolyzed plant-based protein in these models increased the release of growth factors involved in hair growth, specifically Hepatocyte Growth Factor (HGF) and Keratinocyte Growth Factor (KGF).

In another embodiment, a method of prolonging the active growth phase of hair growth comprises contacting the hair and scalp with hydrolyzed plant-based protein.

The hydrolyzed plant-based protein not only provides positive effects on the hair fiber, but surprisingly provided positive effects on the hair follicle.

The hydrolyzed plant-based protein is derived from any part of a plant, including leaves, stems, flowers, fruits, roots, tubers, seeds, a combination thereof, and the like. In a further embodiment, the hydrolyzed plant-based protein is derived from a seed, including a grain, a legume, and the like; a stem, including a tuber, and the like. The hydrolyzed plant-based protein can be obtained from the corresponding plant-based proteins by a chemical, in particular, alkaline or acidic, hydrolysis, by an enzymatic hydrolysis, or a combination of both types of hydrolysis. The hydrolysis of proteins can produce a protein hydrolyzate with a molecular weight distribution of about 100 Daltons to several thousand Daltons.

The hydrolyzed protein can be derived from a legume, for example, black bean, chickpea, cowpea, fava bean, lentil, kidney bean, pea, peanut, pinto bean, soybean, and the like; hemp, potato, rice, wheat, corn, and the like, or a combination thereof.

In an embodiment, the hydrolyzed plant-based protein is derived from a legume, specifically pea.

In an embodiment, the hydrolyzed plant-based protein is derived from potato and wheat.

In an embodiment, the hydrolyzed plant-based protein is derived from hemp and rice.

The hydrolyzed plant-based protein can have a molecular weight, as measured by gel electrophoresis, of about 1 kDa to about 200 kDa, specifically about 3 kDa to about 175 kDa, and more specifically about 5 kDa to about 150 kDa. In an embodiment, the hydrolyzed plant-based protein has a molecular weight peak at about 5 to about 15 kDa, specifically at about 8 to about 12 kDa, and more specifically at about 10 kDa as measured by gel electrophoresis. In an embodiment, the hydrolyzed plant-based protein has a molecular weight peak at about 4 kDa to about 7 kDa, specifically about 5 kDa to about 6 kDa as measured by gel electrophoresis.

In an embodiment, the hydrolyzed plant-based protein comprises amino acids Asp (aspartic acid) in an amount of about 10 to about 15%, Lys (Lysine) at about 5 to about 10%, and Glu (glutamic acid) at about 17 to about 23% based on the total amino acid present in the protein.

The hydrolyzed plant-based protein can be applied to hair, scalp, and hair follicles directly or in the form of a hair treatment composition comprising the hydrolyzed protein, a carrier, and optionally an additive or auxiliary substance used in cosmetic, specifically hair care, formulations. Any type of hair care formulation can be used to deliver the hydrolyzed protein, for example, a tonic, a serum, a shampoo, conditioner, a conditioning shampoo, a hair rinse, a hair mask, or other hair care product formulation. In a specific embodiment, the composition can be formulated as a “leave on” formulation such as a tonic, a serum, and the like, comprising the hydrolyzed protein and a liquid carrier such as water, alcohol (ethanol), and the like to allow for better penetration and to remain on the skin/scalp and not be rinsed off.

The compositions can be used, for example, as creams, foams, lotions, solutions, waters, emulsions such as water-in-oil, oil-in-water, microemulsions, and the like, gels, sprays, and the like. In an embodiment, the composition is a hair tonic, specifically a “leave on” formulation. The hair tonic may be used at room temperature, may have a pH of about 7, and alcoholic content of about 30% to about 35%, or a combination thereof.

Optional additives include a color, an emollient, a humectant (e.g. glycerin, propylene glycol, sorbitol, and the like), an oil, a perfume, a preservative, a surfactant, a vitamin, or a combination thereof.

The compositions comprising the hydrolyzed plant-based protein can be prepared by combining the hydrolyzed plant-based protein with the other composition ingredients, for example, combined with a liquid carrier and an additive.

The user of the methods and compositions described herein can be a mammal, specifically a human. The human can be a male or female.

The hydrolyzed plant-based protein can be formulated into hair treatment compositions comprising about 0.001 to about 10% weight/volume (% w/v), specifically about 0.01 about 7% w/v, more specifically about 0.1 to about 5% w/v, yet more specifically about 0.2 to about 3% w/v, and still yet more specifically about 0.5 to about 1% w/v hydrolyzed plant-based protein in a hair treatment composition. These hair treatment compositions can be used in the methods described herein, e.g., for promoting hair growth, reducing hair loss, prolonging an active growth phase of hair growth of the individual, promoting gene expression of a member of the hair keratin gene family, increasing release of growth factors, or a combination thereof. Within these embodiments, about 0.5 to about 20 ml, specifically about 1 to about 10 ml of hair treatment composition can be applied to the hair and/or scalp of the individual per application.

The frequency of application of the hydrolyzed plant-based protein to an individual in need of treatment can be conducted on an as needed basis, e.g., daily, weekly, or monthly, etc. In an embodiment, the application can be conducted 1, 2, or 3 times a day, once weekly, twice weekly, three times week, or 4 to 6 times a week. The hydrolyzed plant-based protein can either remain on the hair or be rinsed out after a contact time of about 10 seconds to about 48 hours, or more; specifically about 1 minute to about 24 hours, and more specifically about 10 minutes to about 12 hours. The rinsing can involve pure water or water and a shampoo.

In an embodiment, the composition comprising hydrolyzed plant-based protein for use in the methods described herein is free of animal-based proteins and peptides, specifically free of hydrolyzed animal-based protein.

The following examples are merely illustrative of the invention disclosed herein and are not intended to limit the scope thereof.

### EXAMPLES

### Example 1

### Materials & Methods

Studies were conducted on three hydrolyzed plant-based proteins and a comparative hydrolyzed animal-based protein (Table 1A). The amino acid compositions of the hydrolyzed proteins are provided in Table 1B.

TABLE 1A
HYDROLYZED PROTEINS

| Hydrolyzed | |
|---|---|
| proteins | Description |
| Pea | Hydrolyzed protein (pea based) |
| | INCI name: Hydrolyzed Pea Protein |
| | Active content: 100% |
| Potato + | Hydrolyzed protein (potato + wheat based) |
| wheat | INCI name: Aqua (and) Hydrolyzed Vegetable |
| | Protein (and) Hydrolyzed Wheat Protein |
| | Preservatives: Phenoxyethanol & Potassium sorbate |
| Hemp + rice | Hydrolyzed protein (hemp + rice based) |
| | INCI name: Aqua (Water), Hydrolyzed Rice Protein, |
| | Cannabis Sativa Seed Oil |
| | Full INCI: aqua, hydrolyzed hemp seed extract, hydrolyzed |
| | rice protein, citric acid, sodium benzoate, potassium sorbate. |
| Comparative | Hydrolyzed protein (animal keratin based) |
| | INCI name: Aqua (Water), Hydrolyzed Keratin |

TABLE 1B
AMINO ACID COMPOSITION OF
HYDROLYZED PROTEINS

| | Amino acid composition [%] | | | |
|---|---|---|---|---|
| | Pea | Potato/Wheat | Hemp/Rice | Animal |
| | based | based | based | based |
| Cysteic Acid | 0.5-2.5 | 0-0.5 | 0-0.50 | 0.00 |
| Taurine | 0-0.5 | 0-0.5 | 0-0.5 | 0.00 |
| Aspartic Acid | 10-15 | 5-10 | 8-13 | 7.27 |
| Threonine | 1-5 | 2-7 | 2-7 | 6.31 |
| Serine | 2-7 | 2-7 | 2-7 | 9.31 |
| Glutamic Acid | 17-23 | 22-28 | 17-23 | 17.30 |
| Proline | 5-10 | 5-10 | 2-8 | 6.29 |
| Glycine | 3-8 | 1-7 | 2-8 | 5.16 |
| Alanine | 2-7 | 1-7 | 2-8 | 5.38 |
| Valine | 2-7 | 2-8 | 2-8 | 6.69 |
| Cystine | 0-1 | 0.5-2 | 0.1-1.5 | 2.36 |
| Methionine | 0.3-1.5 | 1-3 | 0.5-1.5 | 0.12 |
| Isoleucine | 2.0-6 | 1-7 | 2-8 | 3.80 |
| Leucine | 5-11 | 5-10 | 5-10 | 9.56 |
| Tyrosine | 1.5-4 | 1-7 | 1-7 | 2.40 |
| Phenylalanine | 2-8 | 2-8 | 2-8 | 3.28 |
| Ornithine/ | 0-0.5 | 0-0.5 | 2-8 | 0.86 |
| Lanthionin | | | | |
| Lysine | 5-10 | 2-8 | 1-7 | 2.90 |
| Histidine | 1.5-4 | 1-6 | 1-6 | 0.90 |
| Arginine | 3-10 | 1-7 | 2-8 | 10.10 |

A molecular weight assessment was conducted on the hydrolyzed pea protein by gel electrophoresis. The gel was loaded with molecular weight standard and 3 different concentrations of hydrolyzed pea protein in duplicate. Data show a broad range of protein fragments from 148 kDa to <6 kDa, and two intensity peaks at 10 kDa and at <6 kDa.

### Hair Follicle Models

The analyses were performed with standardized, reconstructed hair follicle models made of keratinocytes, fibroblasts and pseudopapilla (reconstructed dermal papilla composed of microcarriers covered with dermal papilla cells). After final completion of the models, the test substance was applied systemically in non-toxic concentrations.

For determination of gene expression, the RNA was isolated after 24 hours of treatment. All treatments were performed in triplicate.

Release of proteins into the culture media was quantified after 72 hours of incubation. Untreated models served as control and all treatments were performed in triplicate.

### Example 2. Gene Expression Analysis with Real-Time PCR

The gene activity of hair-specific keratins was analyzed after treatment of the reconstructed hair follicle models using the quantitative RT PCR technology. Following incubation, RNA was isolated from hair follicle models using the RNeasy Mini Kit (Qiagen) according to the manufacturer's instructions with slight optimization steps. Spectrometric quantification of the isolated RNA has been performed with Nanodrop-1000 (PeQLab). Following standard processes quantitative RT PCR analysis was performed using a MX3000P RealTime PCR System (Stratagene, La Jolla, California) with a gene specific primer set for KRT33 and KRT34.

After polymerase activation for 10 minutes at 95° C. iterative cycles were set up as follows: 30 seconds at 95° C., 1 minute at 58° C. and 30 seconds at 72° C. Sample values were calculated relatively to a standard RNA from independent hair follicle models. Samples of each model have been analyzed individually.

For quantification of gene expression levels, the untreated control was set to 1 and the samples calculated in ratio to the control (fold-change). Expression values of 1.8 or greater are considered as significant. The results of the gene expression analysis are provided in Table 2.

TABLE 2
RESULTS PCR: PERFORMED ON
HAIR FOLLICLE MODELS

| | | Relative | |
|---|---|---|---|
| | | gene expression | |
| Treatment | Concentration | KRT33A | KRT34 |
| Untreated | — | 1 | 1 |
| Hydrolyzed protein | 0.05% | 2.3 | 4.7 |
| (pea based) | (equals 0.5% | | |
| | animal keratin) | | |
| Hydrolyzed protein | 0.25% | 1.3 | 2.6 |
| (potato + wheat based) | (equals 0.5% | | |
| | animal keratin) | | |
| Hydrolyzed protein | 0.71% | 2.0 | 4.0 |
| (hemp + rice based) | (equals 0.5% | | |
| | animal keratin) | | |
| Hydrolyzed protein | 0.5% | 2.4 | 4.6 |
| (animal keratin based) | | | |

The hair structure is essentially dependent on the composition of special hair-specific structural proteins, the hair keratins. By influencing the composition of these specific proteins, the hair structure can be influenced on a biological level.

### Example 3. Determination of Growth Factors

HGF and KGF are important growth factors released by the dermal papilla to steer different follicular processes such as cell division. HGF is maximum expressed during anagen phase, stimulates hair follicle growth, and plays an important role regarding hair cycle control. Additionally, HGF can retard the anagen-catagen transformation, thus prolonging the active growth phase and counteracting premature hair loss (Jindo T. et al, 1998, Lindner et al., 2000).

KGF is an important factor for healthy growth of the hair follicle, because of its influence on cell division rate and differentiation of keratinocytes (Danilenko et al, 1995, Jang, 2005).

The quantitative analysis of HGF and KGF has been carried out using the suspension bead array technology (Bio-Rad, München) which is based on the principle of a sandwich ELISA. A specific primary antibody, covalent coupled to a color-coded polystyrene bead, binds to the target protein within the sample. This complex could be detected with a specific fluorescent labeled secondary antibody. Using laser scan technology (BioPlex, Bio-Rad) the color-coded beads could be matched to the bound analytes and the amount could be detected fluorometrically. For statistical evaluation at least 50 beads from each color and each sample are measured. Thus the result is equivalent to 50 conventionally performed ELISA assays.

For measurement of HGF and KGF supernatant of the treated hair follicle models has been diluted and analyzed. The results of the determination of growth factors are provided in Table 3.

TABLE 3
RESULTS BIOPLEX: PERFORMED ON
HAIR FOLLICLE MODELS

| | | Relative | |
|---|---|---|---|
| | | protein release | |
| Treatment | Concentration | HGF | KGF |
| Untreated | — | 100 | 100 |
| Hydrolyzed protein | 0.05% | 114.6 | 128.5 |
| (pea based) | (equals 0.5% | | |
| | animal keratin) | | |
| Hydrolyzed protein | 0.25% | 99.3 | 107.0 |
| (potato + wheat based) | (equals 0.5% | | |
| | animal keratin) | | |
| Hydrolyzed protein | 0.71% | 82.9 | 101.7 |
| (hemp + rice based) | (equals 0.5% | | |
| | animal keratin) | | |
| Hydrolyzed protein | 0.5% | 92.6 | 90.8 |
| (animal keratin based) | | | |

### Example 4. Organ Culture of Micro Dissected Human Hair Follicles (Philpott Model

Hair follicles (N=15-25) are prepared from scalp biopsies (from plastic surgery), preserving the dermal papilla and root sheaths, and transferred individually to 24-well microtiter plates for further cultivation. After 24 hours of equilibration in medium, treatment with the test substance dissolved in medium starts (day 0). At that time, the initial length of the follicle is determined using a digital microscope and hair follicles are photographed and measured at each measurement time point (day 0 up to day 9). The length increase is calculated as the difference between the value at the time of measurement and the starting value.

The morphological assessment of anagen and catagen phase of each hair follicle is carried out at the same timepoints. The anagen phase represents the active growth status of the hair, whereas the catagen phase marks the transition to the resting phase.

At the start of the test, intact, anagen hair follicles are selected; an untreated control serves as a reference. The results of the study are provided in Table 4 (hair shaft elongation) and Table 5 (hair follicle phase).

TABLE 4
RESULTS ON MICRO DISSECTED HAIR FOLLICLES (PHILPOTT MODEL):
HAIR SHAFT ELONGATION

| | Hair shaft elongation [μM] | | | | | | | | | |
|---|---|---|---|---|---|---|---|---|---|---|
| | Day 1 | | Day 2 | | Day 5 | | Day 7 | | Day 9 | |
| | MV | SEM | MV | SEM | MV | SEM | MV | SEM | MV | SEM |
| Untreated | 347 | 76 | 463 | 55 | 981 | 118 | 1257 | 166 | 1353 | 199 |
| Hydrolyzed | 341 | 43 | 576 | 58 | 1291 | 130 | 1627 | 173 | 1931 | 203 |
| protein (pea | | | | | | | | | | |
| based) | | | | | | | | | | |

*MV = mean value; SEM = standard error of the mean

TABLE 5
RESULTS MICRO DISSECTED HAIR FOLLICLES
(PHILPOTT MODEL): HAIR FOLLICLE PHASE

| | Hair follicle phase | |
|---|---|---|
| | Anagen | Catagen |
| Untreated | 55% | 45% |
| Hydrolyzed protein (pea based) | 78% | 22% |

### Example 5. Upregulation of Keratin Associated Proteins (KAP) with Hydrolyzed Pea Protein

Hair keratin-associated proteins (KAP or KRTAP) are a major component of the hair fiber and play crucial roles in forming a strong hair shaft through a cross-linked network with keratin intermediate filaments (KIF), which are produced from hair keratins. More than 80 human KAP genes have been identified (Shimomura et al. 2005). Table 6. reports the type of KAP proteins in different regions of hair fiber.

TABLE 6

| KAP | Chemical Group | Location in hair |
|---|---|---|
| KAP1 | High sulfur | Hair cortex |
| KAP2 | High sulfur | Hair cortex |
| KAP3 | High sulfur | Hair cortex |
| KAP4 | Ultrahigh sulfur | Hair cortex |
| KAP6 | High | Hair cortex |
| | glycine-tyrosine | |
| KAP7 | High | Hair cortex |
| | glycine-tyrosine | |
| KAP9 | Ultrahigh sulfur | Hair cortex |
| KAP19.1 | High | Hair cortex |
| | glycine-tyrosine | |
| KAP19.2 | High | Hair cortex |
| | glycine-tyrosine | |
| KAP5 | Ultrahigh sulfur | Hair cuticle |
| KAP10 | High sulfur | Hair cuticle |
| KAP12 | High sulfur | Hair cuticle |
| KAP13.2 | High sulfur | Hair cuticle |
| KAP15 | High sulfur | |
| KAP17.1 | Ultrahigh sulfur | Hair cuticle |
| KAP19.1 | High | Hair cuticle |
| | glycine-tyrosine | |
| KAP19.4 | High | Hair cuticle |
| | glycine-tyrosine | |
| KAP19.6 | High | Hair cuticle |
| | glycine-tyrosine | |
| KAP21 | High | Hair cuticle |
| | glycine-tyrosine | |
| KAP21.2 | Ultrahigh sulfur | Hair cuticle |
| KAP23 | High | Hair cuticle |
| | glycine-tyrosine | |
| KAP8 | High | Hair matrix/ |
| | glycine-tyrosine | cortex cell region |
| KAP11 | High sulfur | Hair matrix/ |
| | | cortex cell region |
| KAP13 | High sulfur | Hair matrix/ |
| | | cortex cell region |
| KAP15 | High sulfur | Hair matrix/ |
| | | cortex cell region |
| KAP19.3 | High | Hair matrix/ |
| | glycine-tyrosine | cortex cell region |
| KAP19.6 | High | Hair matrix/ |
| | glycine-tyrosine | cortex cell region |
| KAP19.7 | High | Hair matrix/ |
| | glycine-tyrosine | cortex cell region |
| KAP20 | High | Hair matrix/ |
| | glycine-tyrosine | cortex cell region |
| KAP21 | High | Hair matrix/ |
| | glycine-tyrosine | cortex cell region |
| KAP21.2 | Ultrahigh sulfur | Hair matrix/ |
| | | cortex cell region |
| KAP23 | High sulfur | Hair matrix/ |
| | | cortex cell region |

In a volunteer study using plucked hair follicles, 71 KAPs increased by log2PC of approximately 0.4 (Table 7, selection of KAPs) and cover all three chemical groups and also all synthesis sites of the hair follicle. Transcriptomic changes by the treatment were quantified by means of RNA sequencing.

TABLE 7
(SELECTION)

| | log2FC, |
|---|---|
| KAP | after treatment |
| KAP9-9 | 0.43 |
| KAP9-8 | 0.44 |
| KAP9-4 | 0.41 |
| KAP9-2 | 0.48 |
| KAP5-8 | 0.45 |
| KAP5-7 | 0.42 |
| KAP5-5 | 0.41 |
| KAP5-2 | 0.44 |
| KAP5-11 | 0.46 |
| KAP5-10 | 0.48 |
| KAP5-1 | 0.38 |
| KAP4-8 | 0.44 |
| KAP4-7 | 0.42 |
| KAP4-6 | 0.42 |
| KAP4-5 | 0.45 |
| KAP4-4 | 0.45 |
| KAP4-3 | 0.43 |
| KAP4-12 | 0.48 |
| KAP4-11 | 0.45 |
| KAP4-1 | 0.42 |
| KAP3-3 | 0.41 |
| KAP3-2 | 0.41 |
| KAP3-1 | 0.43 |
| KAP26-1 | 0.28 |
| KAP24-1 | 0.28 |
| KAP21-2 | 1.34 |
| KAP2-4 | 0.44 |
| KAP2-3 | 0.46 |
| KAP2-2 | 0.48 |
| KAP2-1 | 0.43 |
| KAP17-1 | 0.32 |
| KAP12-2 | 0.38 |
| KAP12-1 | 0.35 |
| KAP10-9 | 0.51 |
| KAP10-8 | 0.45 |
| KAP10-7 | 0.45 |
| KAP10-5 | 0.41 |
| KAP10-4 | 0.45 |
| KAP10-3 | 0.41 |
| KAP10-2 | 0.46 |
| KAP10-12 | 0.5 |
| KAP10-11 | 0.4 |
| KAP10-10 | 0.44 |
| KAP10-1 | 0.42 |
| KAP1-5 | 0.43 |
| KAP1-4 | 0.37 |
| KAP1-3 | 0.44 |
| KAP1-1 | 0.43 |

The literature reports the decrease of KAP gene expression with age in plucked hair follicles in KAP4-3, KAP4-4, KAP4-7, KAP9-8, and KAP12-2 (Giesen et al. 2011). Gene expression of KAP proteins in plucked hair follicles after treatment with hydrolyzed pea protein were upregulated (Table 8).

TABLE 8

| KAP | log2FC, after treatment |
|---|---|
| KAP4-3 | 0.43 |
| KAP4-4 | 0.45 |
| KAP4-7 | 0.42 |
| KAP9-8 | 0.44 |
| KAP12-2 | 0.38 |

KAP4-3, 4-4, 4-7, 9-8, and 12-2 are downregulated with age yet are upregulated through hydrolyzed pea protein treatment. These data support the use of hydrolyzed plant-based protein, specifically hydrolyzed pea protein, to counteract the aging process of hair, promote the formation of a stronger hair shaft, and promote better hair growth.

### Example 6. Upregulation of Collagen Gene COL15A1 with Hydrolyzed Pea Protein

It has been reported in the literature that the expression of collagen genes COL13A1 and COL15A1 are downregulated in aged hair follicles, and aged dermal papilla cells were difficult to aggregate, which decreases hair inductivity, inducing senescence (Kim et al. 2021). In an assay, hydrolyzed protein (pea based) 0.05% was found to upregulate Col15A1 by log2FC of 1.48 (RNA sequencing method). These results support the anti-aging effect of hydrolyzed plant-based proteins on hair.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A method of promoting hair growth and reducing hair loss, comprising:
contacting hair and/or scalp of an individual with an effective amount of a hydrolyzed plant-based protein.

2. The method of claim 1, wherein the hydrolyzed plant-based protein is derived from a seed, a legume, a stem, or a combination thereof.

3. The method of claim 1, wherein the hydrolyzed plant-based protein is derived from a legume, hemp, potato, rice, wheat, corn, or a combination thereof.

4. The method of claim 1, wherein the hydrolyzed plant-based protein is hydrolyzed pea protein, hydrolyzed potato and wheat protein, or hydrolyzed hemp and rice protein.

5. The method of claim 1, wherein the hydrolyzed plant-based protein is hydrolyzed pea protein.

6. The method of claim 1, wherein the hydrolyzed plant-based protein comprises amino acids Asp (aspartic acid) in an amount of about 10 to about 15%, Lys (Lysine) at about 5 to about 10%, and Glu (glutamic acid) at about 17 to about 23% based on the total amino acids present in the protein.

7. The method of claim 1, wherein the hydrolyzed plant-based protein is formulated into a hair treatment composition comprising about 0.001 to about 10% w/v, specifically about 0.01 about 7% w/v, more specifically about 0.1 to about 5% w/v, yet more specifically about 0.2 to about 3% w/v, and still yet more specifically about 0.5 to about 1% w/v hydrolyzed plant-based protein.

8. The method of claim 7, wherein about 0.5 to about 20 ml, specifically about 1 to about 10 ml of hair treatment composition is applied per application.

9. The method of claim 1, wherein the hydrolyzed plant-based protein is applied to the individual once daily, twice daily, three times daily, once weekly, twice weekly, three times week, or 4 to 6 times a week.

10. The method of claim 1, further comprising
prolonging an active growth phase of hair growth of the individual;
promoting gene expression of a member of the hair keratin gene family;
increasing release of growth factors;
or a combination thereof.

11. A method of promoting gene expression of a member of the hair keratin gene family, increasing release of growth factors, or a combination thereof, comprising:
contacting hair and/or scalp of an individual with an effective amount of a hydrolyzed plant-based protein.

12. The method of claim 11, wherein the hair keratin gene is KRT33A, KRT34, or a combination thereof.

13. The method of claim 11, wherein the growth factor is Hepatocyte Growth Factor, Keratinocyte Growth Factor, or a combination thereof.

14. A method of prolonging the active growth phase of hair growth, comprising:
contacting hair and/or scalp of an individual with an effective amount of a hydrolyzed plant-based protein.

15. A method of promoting gene expression of one or more hair keratin-associated proteins or collagen gene, comprising:
contacting hair and/or scalp of an individual with an effective amount of a hydrolyzed plant-based protein;
wherein the hair keratin-associated proteins is KAP4-3, KAP4-4, KAP4-7, KAP9-8, KAP12-2, or a combination thereof; and the collagen gene is COL13A1.
